(19) United States
(12) Patent Application Publication
(10) Pub. No.: US 2021/0393446 A1
(43) Pub. Date: Dec. 23, 2021

(54) UNIQUE OPTICAL SIGNALS FOR SENSOR DETECTION IN ABSORBENT ARTICLES

(71) Applicant: The Procter & Gamble Company, Cincinnati, OH (US)

(72) Inventors: Blanca Arizti, Brooklyn, NY (US); Christian M. Best, Cincinnati, OH (US)

(21) Appl. No.: 16/904,936

(22) Filed: Jun. 18, 2020

(51) Int. Cl. A61F 13/42

(52) U.S. Cl. CPC A61F 13/42; A61F 2013/429; A61F 2013/422

(57) ABSTRACT

A system includes an absorbent article that absorbs and contains bodily exudates and comprises an indication unit that generates an optical signal indicative of presence of the bodily exudates within the absorbent article. The indication unit comprises an optical property changing element that changes from having a first optical property to having a second optical property in response to exposure to the bodily exudates. The indication unit further comprises a visual modifying element proximate that provides a visual modification to the second optical property, resulting in the optical signal comprising a third optical property, wherein a sensor device that attaches to the absorbent article detects the presence of the bodily exudates based on recognition of the optical signal comprising the third optical property.

### TECHNICAL FIELD

This application relates to sensor systems that integrate unique optical indicators on or within absorbent articles for detection by a sensor device located on or near the absorbent articles.

### BACKGROUND

Absorbent articles for personal hygiene, such as disposable diapers for infants, training pants for toddlers or adult incontinence undergarments are designed to absorb and contain bodily exudates, in particular, large quantities of urine. These absorbent articles comprise several layers providing different functions, for example a topsheet, a backsheet and in-between an absorbent core, among other layers. The function of the absorbent core is to absorb and retain the exudates for a prolonged amount of time, for example overnight for a diaper, minimize re-wet to keep the wearer dry and avoid soiling of clothes or bed sheets.

It has been proposed to incorporate sensors into absorbent articles to facilitate sensing usage information (e.g., timing of initiation and level of saturation associated with urination and/or defecation) and/or providing notifications to users (e.g., caregivers, article manufacturers, etc.) regarding the usage information. However, it is believed that improvements are still necessary for such sensor systems.

For example, some sensor devices have been proposed for detecting wetness or other conditions of an absorbent article based on a chemical-based indicator that changes color in response to exposure to bodily exudates. Typically, in wetness indicators, the color change is generally from a yellow color to blue or green color. It is difficult to change the chemical composition of such color changing wetness indicators. Accordingly, any absorbent article that incorporates a conventional color changing indicator can conceivable be used with a sensor device configured to detect wetness etc. based on the conventional color changing indicator, enabling competitors to develop and sell off-brand or counterfeited products. Consequently, manufacturers desire mechanisms to mitigate use of proprietary sensor devices with absorbent articles produced by other manufactures that have not been safely cleared for use, as well as mechanisms to ensure and track authorized use of their products for providing loyal customers with ancillary services.

### SUMMARY

The invention comprises the features of the independent claims herein. A system comprises an absorbent article that absorbs and contains bodily exudates, and an indication unit formed on or within the absorbent article that generates an optical signal indicative of the presence of the bodily exudates. The indication unit comprises an optical property changing element that changes from having a first optical property to having a second optical property in response to exposure to the bodily exudates. The indication unit further comprises a visual modifying element proximate to the optical property changing element that provides a visual modification to the second optical property, resulting in the optical signal comprising a third optical property.

A sensor device comprises a detection unit that detects presence of bodily exudates in an absorbent article based on detection of an optical signal with a defined optical property generated by an indication unit of the absorbent article. The defined optical property is based on a combination of an optical property changing element of the indication unit that changes from having a first optical property to having a second optical property in response to exposure to the bodily exudates, and a visual modifying element proximate to the optical property changing element that provides a visual modification to the second optical property.

A system comprise an absorbent article configured to absorb and contain bodily exudates and an indication unit formed on or within the absorbent article. The indication unit comprises an optical property changing element that changes from having a first optical property to having a second optical property in response to exposure to the bodily exudates; and a visual modifying element proximate to the optical property changing element that provides a defined visual mark. The system further comprises a sensor device that removably attaches to the absorbent article and detects presence of the bodily exudates in the absorbent article based on detection of a defined optical property that results from a combination of the second optical property and the defined visual mark.

Elements described in the disclosed systems can be embodied in different forms such as a computer-implemented method, a computer program product, or another form.

### DESCRIPTION OF THE DRAWINGS

FIG. 1 illustrates a block diagram of an example non-limiting system that integrates one or more optical signals on or within absorbent articles for sensor detection in accordance with one or more embodiments of the disclosed subject matter.

FIGS. 2A and 2B present a schematic illustration of an example sensor device in accordance with one or more embodiments of the disclosed subject matter.

FIGS. 3A and 3B illustrate example indication units of an absorbent in accordance with one or more embodiments of the disclosed subject matter.

FIG. 4 illustrates generation and detection of a distinct optical signal based on combination of an optical property changing element and a visual modifying element in accordance with one or more embodiments of the disclosed subject matter.

FIG. 5 presents some example optical property changing element and visual modifying element combinations that can be used to generate distinct optical signals in accordance with one or more embodiments of the disclosed subject matter.

FIG. 6 presents some additional example optical property changing element and visual modifying element combinations that can be used to generate distinct optical signals in accordance with one or more embodiments of the disclosed subject matter.

FIG. 7 illustrates a block diagram of an example sensor device configured to detect property(ies) of an absorbent article based on recognition of an optical signal generated by an indication unit having a defined optical property, in accordance with one or more embodiments of the disclosed subject matter.

FIG. 8 illustrates a block diagram of an example external user device that facilitates determining and reporting property(ies) of an absorbent article based on recognition of an optical signal generated by an indication unit having a defined optical property in accordance with one or more embodiments of the disclosed subject matter.

FIG. 9 presents a high-level flow diagram of another example process for detecting property(ies) of an absorbent article based on recognition of an optical signal generated by an indication unit having a defined optical property in accordance with one or more embodiments of the disclosed subject matter.

### DETAILED DESCRIPTION

The following detailed description is merely illustrative and is not intended to limit embodiments and/or application or uses of embodiments.

“Absorbent article” as used herein refers to a variety of devices which are placed or worn against or in proximity to the body of the wearer to absorb and contain various exudates discharged from the body, such as disposable diapers. Typically, these absorbent articles comprise a topsheet, a backsheet, an absorbent core and optionally an acquisition system (which may be comprised of one or several layers) and other components, with the absorbent core normally placed between the backsheet and the acquisition system or topsheet. The function of the absorbent core is to absorb and retain the exudates. Although various embodiments of the disclosed subject matter are exemplified in association with the absorbent article being a disposable diaper, it should be appreciated that the disclosed techniques can be applied to a variety of other types of absorbent articles, including reusable diapers (e.g., cloth diapers), absorbent inserts which may be disposable or reusable and may be used in combination with reusable outer covers, pants, training pants, pads, adult incontinence products, and/or feminine hygiene products (including, for example, sanitary napkins and tampons).

“Sensor device” refers to any electrical device that can be attached to and/or integrated on or within an absorbent article that provides for capturing and/or generating sensory feedback data associated with wear of the absorbent article via one or more sensors formed on or within the sensor device. In various embodiments, the sensor device can be configured to removably attach to disposable diapers and/or other absorbent articles.

“Sensory feedback data” (or simply “sensory feedback”) as used herein refers to any type of data captured by one or more sensors formed on or within the sensor device and/or determined or inferred based on the captured sensor data. In this regard, unless context warrants particular distinctions among the terms, sensory feedback data can include raw sensor measurements (e.g., raw color sensor data, raw motion sensor measurements, etc.) and/or processed feedback information determined based on the raw sensor measurements using one or more algorithms, heuristics, machine learning models, etc. (e.g., a determined saturation/wetness level, a determined activity level, etc.). Sensory feedback data includes usage data and/or activity data.

“Usage” in relation to information captured, to be captured, inferred/determined from information captured, processed, detected, stored and/or transmitted, or otherwise used in the sensor systems described herein refers information regarding occurrence and/or timing of the exudation events (e.g., urination, defecation), amount of bodily exudates (e.g., by volume, by weight) associated with an exudation event, saturation levels, time to saturation of the absorbent article, loading status, amount of bodily exudates contained within the absorbent article over a period of time, frequency of exudation events, frequency of article changes, duration of exposure time to bodily exudates, type of the bodily exudates (e.g., urine, feces, discharge, etc.), characteristics of the bodily exudates (e.g., runny bowel, mushy/pasty bowels, viscosity of exudates, coloration of the exudates, etc.), biomarkers present in the bodily exudates, and/or other details related to the use of an absorbent article.

“Activity” in relation to information captured, to be captured, inferred/determined from information captured, processed, detected, stored and/or transmitted, or otherwise used in the sensor systems described herein refers to information regarding exertion levels, movement, exertion and/or movement patterns, sleep/wake patterns, positions, motions, defined movements and motions (e.g., laying, laying on back, laying on stomach, sitting, kicking, walking, crawling, grabbing/pulling on diaper, etc.), and/or other details related to the actions of the wearer during wear of an absorbent article.

“Joined” or “attached” as used herein encompasses configurations whereby an element is directly secured to another element by affixing the element directly to the other element, and configurations whereby an element is indirectly secured to another element by affixing the element to intermediate member(s) which in turn are affixed to the other element. The terms further include embodiments in which a pocket or other connector is formed in or attached to an area of the absorbent article. Further, these terms include configurations in which the elements are removably or non-removably attached.

“Processor” as used herein refers to a device that executes machine/computer executable instructions or components stored in memory. A processor as used herein includes, but is not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize use of space or enhance performance of user equipment. A processor can also be implemented as a combination of computing processing units.

“Component” as it relates to a sensor device, a system incorporating a sensor device and/or other machinery herein refers to a computer-related entity or an entity related to an operational machine with one or more specific functionalities. The entities may be hardware, software, a combination of hardware and software, or software in execution. One or more components can reside within a process and/or thread of execution and a component can be localized on one computer and/or distributed between two or more computers. Components can communicate via local and/or remote processes. A component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system. Embodiments of systems described herein can include one or more machine-executable components embodied within one or more machines (e.g., embodied in one or more computer-readable storage media associated with one or more machines). Such components, when executed by the one or more machines (e.g., processors, computers, computing devices, virtual machines, etc.) can cause the one or more machines to perform the operations described.

“Memory” as used herein refers to mechanism(s) used to retain information, such as executable instructions or components. As used herein, terms such as “store,” “storage,” “data store,” data storage,” “database,” and substantially any other information storage element relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

The disclosed subject matter is directed to sensor systems that integrate one or more optical signals on or within absorbent articles for detection of chemical, biological and/or physical changes to the absorbent articles via a sensor device positioned proximate to the optical signal(s). In one or more embodiments, the disclosed techniques combine a visual modifying element with a conventional color changing indicator to produce a unique optical signal that is detected by the sensor device. The sensor device can further be configured to send notification to an external device communicatively coupled to the sensor device (e.g., a paired device) alerting a user of the external device (e.g., the wearer of the absorbent article, a caregiver of the wearer, etc.) of usage information such as the presence of exudates (e.g., urine, feces, discharge, menses) based on detection of the unique optical signal. In this regard, the sensor device can further be configured to only detect and/or report said usage information in response to detecting and recognizing the unique optical signal, thereby ensuring the sensor device only works with absorbent articles comprising the unique optical signal, and vice versa.

In some embodiments, the unique optical signal can be formed by an overlapping layer at least partially superposed with a conventional color-changing indicator, wherein the overlapping layer changes the appearance/color of the conventional indicator via, for example, a filtering effect. Additionally, or alternatively, the absorbent article can incorporate a defined visual mark, such as a pattern, symbol, code, image, watermark or the like, on or near the conventional color-changing indicator. With these embodiments, the sensor device can be configured to only detect and/or report usage information based on detection of a combination of the color change of the conventional color-changing indicator and defined visual mark.

One or more embodiments are now described with reference to the drawings. In the following description, numerous specific details are set forth in order to provide a more thorough understanding of the one or more embodiments. It is evident, however, in various cases, that the one or more embodiments can be practiced without these specific details. In addition, certain elements may be left out of particular views for the sake of clarity and/or simplicity when explanations are not necessarily focused on the omitted elements.

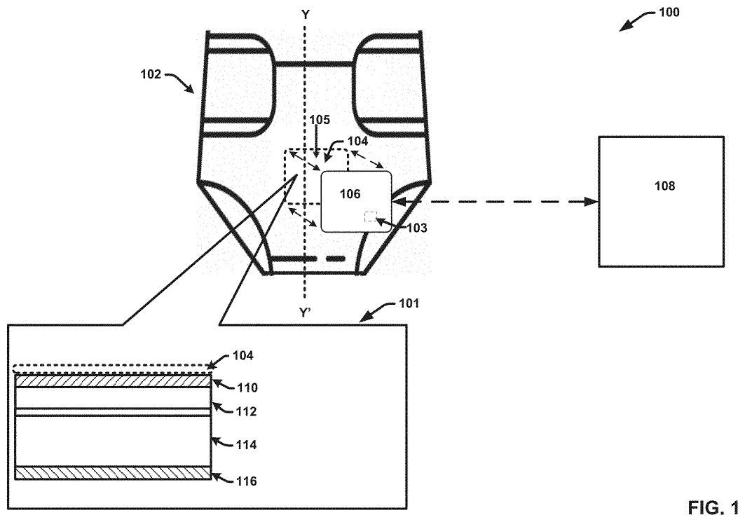

FIG. 1 illustrates a block diagram of an example, non-limiting system 100 that integrates one or more unique optical signals on or within absorbent articles for detecting changes in the absorbent articles by a sensor device proximate to the optical signals(s). System 100 includes an absorbent article 102, a sensor device 106 and an external user device 108. In the embodiment shown, the absorbent article 102 is a diaper, such as a disposable diaper. However, it should be appreciated that the absorbent article 102 can be or include a variety of other types of absorbent articles.

In one or more embodiments, the sensor device 106 can be configured to removably attach to the absorbent article 102 and capture and/or generate sensory feedback data associated with wear of the absorbent article 102 using one or more sensors 103 formed on or within a housing (described infra) of the sensor device 106. Although system 100 depicts a single absorbent article 102 for use with the sensor device 106, the sensor device 106 can be designed to be reused with and reattached to a plurality of absorbent articles. In various embodiments, the sensor device 106 can be configured to removably attach to the absorbent article 102 at or near an attachment zone 104 disposed on exterior portion of the absorbent article 102. The location of the attachment zone 104 can vary depending on the type of the absorbent article 102, the type of sensory feedback data the sensor device 106 is configured to capture/detect, and/or the mechanism via which the sensor device 106 captures/detects the sensory feedback data in association with attachment to the absorbent article 102.

Nonlimiting examples of sensors 103 include one or more optical sensors, motion sensors (e.g., an accelerometer, a gyroscope, etc.), image sensors, biosensors, biochemical sensors, temperature sensors, force/pressure sensors, humidity sensors, acoustic sensors, an RFID reader/sensor, biofeedback sensors configured to detect physiological parameters associated with the wearer (e.g., heart rate, temperature, and other vital signs, biomarkers present in bodily exudates and the like), and the like. Additionally, or alternatively, at least one sensor can be configured to sense, detect or otherwise capture sensor data reflective on a behavior of the wearer, pressure and irritation associated with fit and/or wear of the absorbent article, and the like. Further, the activity information can include motion and/or movement data (e.g., captured via one or more motion sensors) that can be correlated to defined bodily movements and/or positions, movement patterns, activity patterns (e.g., sleep/wake patterns), activity levels, and the like.

For example, in accordance with various embodiments described herein, using one or more optical sensors, the sensor device 106 is configured to monitor and detect usage information (e.g., presence of exudates, wetness level) based on changes in an optical state of an indication unit 112 located at or near a crotch region of the absorbent article 102. With these embodiments, the attachment zone 104 can be located at or near the crotch region at a position that aligns the one or more optical sensors 206 with the indication unit 112.

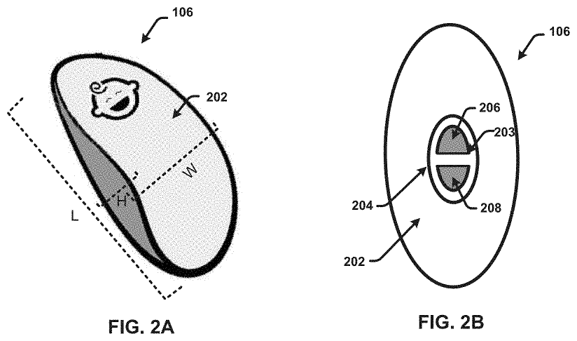

In this regard, FIGS. 2A and 2B present a schematic illustration of an exemplary sensor device 106. FIG. 2A presents a three-dimensional view of top (garment-facing) and side surfaces of the housing 202. FIG. 2B presents a two-dimensional view the backside (wearer-facing side) of the sensor device 106, wherein the backside of the sensor device opposes the topside of the housing that includes the baby image/symbol as shown in FIG. 2A, and wherein the backside of the housing 202 is adapted to be attached to and face the attachment zone 104 of the absorbent article.

With reference to FIGS. 2A and 2B in view of FIG. 1, in various embodiments, the housing 202 and/or the absorbent article 102 can include one or more connectors 105 for removably attaching the sensor device 106 to the absorbent article 102 at or near the attachment zone 104. The connectors can be provided such that the sensor device 106 can be attached to and detached from the absorbent article 102. The connectors can also allow for refastening of the sensor device 106 to the absorbent article 102 at or near the attachment zone 104.

The connectors 105 can include one or more adhesives or cohesives formed on the attachment zone 104 and/or on the backside of the housing 202. Such connectors can further include one or more mechanical fasteners, including strap-based fasteners, hook-and-loop-fasteners (e.g., Velcro™), or fasteners comprising at least one button or at least one magnet. In another implementation, a pocket can be formed at or near the attachment zone 104 of the absorbent article 102 and the sensor device 106 can be inserted into the pocket. For example, some absorbent articles can be provided as pants comprising a crotch portion and a belt portion. The crotch portion and the belt portion can be joined adhesively or mechanically. In the area of adhesive joining, a certain portion can be free of adhesive and accessible from the outside. This portion can then serve as a pocket for receiving the sensor device 106. A belt, strap or other device may be used to place and hold the sensor device 106 relative to the absorbent article 102. The sensor device 106 can similarly be joined or held to an article of clothing worn by the wearer of the absorbent article.

The size, shape, and/or dimensions of the housing 202 can vary. In some implementations in which the sensor device 106 is designed to be removably attached to disposable diapers, for safety (so as to not become a choking hazard) and convenient handling, the housing 202 can have a length (L) of at least 1.0 centimeters (cm), 2.0 cm, 3.0 cm, 4.0 cm or more (but normally less than 15.0 cm), a width (W) of least 1.0 cm, 2.0 cm, 3.0 cm or more (but normally less than 15.0 cm), and a height (H) of at least 0.5 centimeters, 1.0 cm, 2.0 cm, 3.0 cm, 4.0 cm or more (but normally less than 15.0 cm).

The material employed for the housing 202 can also vary. In some implementations, the housing 202 can be formed with a rigid material (e.g., a rigid plastic). In other implementations, the housing 202 can be formed with a flexible or partially flexible material. To be flexible, the sensor device 106 can incorporate flexible electronic components (and boards). Some suitable materials for the housing 202 can include but are not limited to, silicon, plastic, a thermoplastic, a thermoplastic elastomer (TPE), a confection, a thermosetting polymer, rubber, and the like.

The sensor device 106 includes a detection unit 204 configured to detect sensory feedback data from one or more sensors 203, having any of the features or functionalities of the sensor 103 described above and vice versa. In the embodiment shown in FIG. 2B, the detection unit 204 is configured to detect optical property changes reflected in the indication unit 112 when the sensor device is attached to the absorbent article at or near the attachment zone 104. The optical property changes can reflect a chemical, biological and/or physical change to the absorbent article which may correlate to usage information. For example, the optical property changes can reflect the presence of bodily exudates, wetness level, volume of bodily exudates, pH changes, type(s) of the bodily exudates, biomarkers and the like. In accordance with these embodiments, the absorbent article 102 can comprise an indication unit 112 formed on or within the absorbent article 102 that is configured to change an optical property thereof in response a change to the article (e.g., wetness, a pH change) and provide a distinct optical signal indicative of the change. In this regard, the detection unit 204 can be particularly configured to detect the distinct optical signal using one or more optical sensors 206 positioned proximate to the indication unit 112 when the sensor device 106 is attached to the absorbent article 102 at or near the attachment zone 104. The optical sensor(s) 206 are configured to detect coloration changes and/or other visual characteristics of the indication unit 112, including but not limited to: visual patterns, shapes, marks, images, objects, identifiers, codes (e.g., including barcodes, hash codes, alphanumeric codes, etc.), and the like.

For example, call out box 101 presents an enlarged view of a transversal cross-section of a portion of the absorbent article 102 taken along axis Y-Y′ in accordance with some example embodiments. As shown in call out box 101, in some embodiments, the absorbent article 102 can comprise a backsheet 110 that is an outer layer of the absorbent article 102. The absorbent article 102 can further include one or more internal layers 114 formed on or joined to the backsheet 110. Such internal layers 114 may include one or more layers of an acquisition system, one or more layers of an absorbent core, and/or one or more inner layers of the backsheet. The one or more internal layers 114 include the part of the absorbent article 102 configured to absorb and contain bodily exudates. The one or more internal layers 114 can include one or more layers of absorbent material, including but not limited to: a superabsorbent polymer (SAP) material, a cellulosic fiber material, a synthetic fiber material and the like.

The topsheet 116 is the part of the absorbent article 102 intended to be directly in contact with the wearer's skin. The topsheet 116 preferably comprises a soft material that is not irritating to the wearer's skin. Further, at least a portion of the top sheet 116 is liquid permeable, permitting liquids to readily penetrate through its thickness. The topsheet 116 can be manufactured from a wide range of materials, such as porous foams, reticulated foams, aperture plastic films, woven or nonwoven materials of natural fibers (e.g., wood or cotton fibers), synthetic fibers or filaments (e.g., polyester or polypropylene or biocomponent PE/PP fibers or mixtures thereof), or a combination of natural and synthetic fibers.

The backsheet 110 generally includes the portion of the absorbent article 102 to which the sensor device 106 is attached. The sensor device 106 may be attached to the backsheet at or near the attachment zone 104. In non-limiting examples, the backsheet 110 can include the attachment zone 104 printed thereon or otherwise identified thereon. The backsheet also generally prevents the exudates absorbed and contained therein from soiling articles such as bedsheets and undergarments. The backsheet 110 can thus comprise a material that is essentially impermeable to liquids. The backsheet 110 can for example be or comprise a thin plastic film such as a thermoplastic film. Other suitable backsheet materials can include breathable materials which permit vapors to escape from the absorbent article 102 while still preventing exudates from passing through the backsheet 110. Exemplary breathable materials include materials such as woven webs, nonwoven webs, composite materials such as film-coated nonwoven webs, microporous films, and the like. In various embodiments, the backsheet 110 can be formed with a transparent or semitransparent material that provides for exposing an optical signal generated by the indication unit 112 to the detection unit 204 of the sensor device 106.

Components of the absorbent article can at least partially be comprised of bio-sourced content as described in U.S. Pat. Pub. Nos. 2007/0219521A1, 2011/0139658A1, 2011/0139657A1, 2011/0152812A1, and 2011/0139659A1. These components include, but are not limited to, topsheets, backsheet films, backsheet nonwovens, side panels, leg gasketing systems, superabsorbent, acquisition layers, core wrap materials, adhesives, outer covers, fastener systems, and landing zones. In at least one embodiment, an absorbent article component comprises a bio-based content value from about 10% to about 100%, or from about 25% to about 75%, or from about 50% to about 60% using ASTM D6866-10, method B. In order to apply the methodology of ASTM D6866-10 to determine the bio-based content of any component, a representative sample of the component must be obtained for testing. In at least one embodiment, the absorbent article component can be ground into particulates less than about 20 mesh using known grinding methods (e.g., WILEY® mill), and a representative sample of suitable mass taken from the randomly mixed particles.

The absorbent article 102 includes an indication unit 112, which may be formed between the backsheet 110 and the one or more internal layers 114 within a region of the absorbent article that at least partially overlaps the attachment zone 104 to which the sensor device 106 is intended to be attached. For example, in some embodiments, the housing 202 of the sensor device 106 can be adapted to be physically coupled to the absorbent article 102 such that the sensor device 106 is further communicatively coupled to the indication unit 112. In this regard, the indication unit 112 can directly contact at least a portion of the one or more internal layers 114. In the embodiment shown, the indication unit 112 is positioned directly on a garment-facing surface of the one or more internal layers 114. The indication unit 112 or a portion of the indication unit 112 can be formed within the one or more internal layers 114.

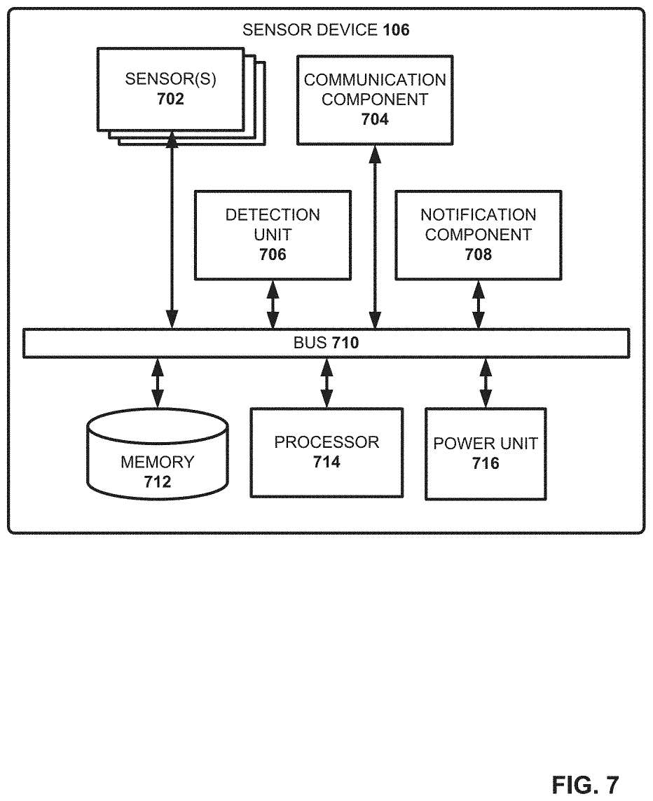

As discussed in greater detail infra, the indication unit 112 provides a distinct optical signal 402 indicative of a particular state (e.g., presence of exudates, wetness level) of the absorbent article 102. Using one or more optical sensors 206 of the detection unit 204, the sensor device 106 monitors the optical state of the indication unit 112 to determine when the indication unit 112 produces the distinct optical signal 402. For example, the sensor device 106 can employ one or more optical sensors that generate a defined output signal based on detection of the distinct optical signal. The one or more optical sensors can provide an output that varies depending on the presence or absence of bodily exudates which depends on the optical state of the indication unit 112. The sensor device 106 can further include suitable electronic circuitry (e.g., hardware), software, or a combination thereof, that provides for processing of raw sensor measurements representative of a measured property (e.g., a distinct optical signal and/or optical state of the indication unit 112) as captured via the one or more sensors of the sensor device 106 into a digital signal corresponding to the measured property. For example, such electronic circuitry can include but is not limited to, excitation control elements, amplification elements, analogue filtering elements, data conversion elements, compensation elements, and the like. As described in greater detail infra with reference to FIG. 7, the sensor device 106 can also include or be operatively coupled to at least one memory that stores computer executable instructions and at least one processor (e.g., a microprocessor) that executes the computer executable instructions stored in the memory.

The sensor device 106 can also include suitable communication hardware and/or software that provides for wireless (and/or wired) communication between the sensor device 106 and at least one external user device 108. The external user device 108 can include essentially any type of computing device capable of at least receiving information from the sensor device 106. For example, the external user device 108 can include but is not limited to: a mobile phone, a smartphone, a smartwatch, a tablet personal computer, a laptop computer, a desktop computer, a video monitoring device (e.g., a video baby monitor device), an audio monitoring device (e.g., an audio baby monitor device), an augmented reality (AR) device, a virtual reality (VR), a heads-up display (HUD), a smart speaker device, another sensor device, an IoT device, a television, an Internet enabled television, and similar types of devices.

For example, in some embodiments, the sensor device 106 can be configured to transmit captured sensor data to an external user device 108 associated with the, a caregiver for the wearer, or another suitable entity for processing and/or presentation to associated individual/entity via a display, speaker, or another suitable output mechanism of the external user device 108. The external user device 108 can be configured to process and/or analyze the sensor data to determine and/or infer sensory feedback data based on captured sensor measurements. For example, the external user device 108 can receive sensor data from the sensor device 106 identifying or indicating a measured property and/or status of at least one indicator 112. In another example, the external user device 108 can receive sensor data including chemical sensor measurements, temperature sensor measurements, motion sensor measurements, pressure sensor measurements, and the like, as captured via corresponding sensors located on or within the sensor device 106. The external user device 108 can further process/analyze the received sensor data using predefined processing logic (e.g., algorithms, heuristics, machine learning models, defined correlations, tracked data correlations, etc.) to determine and/or infer sensory feedback data regarding usage and/or activity. For example, in some embodiments, the external user device 108 can receive sensor data from the sensor device that indicates an optical state or property of the indication unit 112. The external user device can further process the sensor data using predefined processing logic to determine whether the optical state or property of the indication unit 112 corresponds to a distinct optical signal indicative of particular usage information.

In some embodiments, the external user device 108 can further present or otherwise render the sensory feedback information at the external user device 108. For example, in implementations in which the absorbent article 102 is a diaper, based on a determination that the sensor data indicates the diaper is wet, the external user device 108 can generate and render a notification at the external user device notifying the caregiver that the diaper needs changing. In addition to processing and/or rendering the captured sensor data to provide feedback to the user, the sensor device 106 and/or the external user device 108 can also store the sensor data and/or the sensory feedback data determined therefrom in suitable data storage for data aggregation.

Additionally, or alternatively, the sensor device 106 itself can include onboard processing logic for processing some or all of the sensor data to determine and/or infer sensory feedback data based on the captured sensor data measurements. With these embodiments, the sensor device 106 can be configured to transmit the processed sensory feedback data to the external user device 108 for presentation to the device user and/or for further analytical processing (e.g., by the external user device 108, an application server for the connected care system, another system or the like). For example, in various embodiments, the sensor device 106 can be configured to determine whether the indication unit 112 provides the distinct optical signal that indicates the absorbent article is wet (or another defined state of the absorbent article 102). The sensor device 106 can also be configured to notify the external user device 108 regarding the detection of the distinct optical signal and/or the corresponding property represented by the distinct optical. The external user device 108 can in turn render a notification at the external user device 108 (e.g., a visible notification shown via a device display, an audible notification, etc.) notifying a user of the external device that the absorbent article 102 is wet (or other defined state).

In other non-limiting examples, the sensor device 106 can include onboard processing logic that can determine when the absorbent article 102 has reached a threshold saturation level and thus requires changing based on detection of a distinct optical signal provided by the indication unit 112. Based on a determination that the threshold saturation level has been reached, the sensor device 106 can be configured to transmit a notification message to the external user device 108 that indicates the absorbent article 102 has reached the threshold saturation level. The external user device 108 can further render the notification message using an appropriate rendering mechanism (e.g., as a visual notification rendered via a display, as an audible alarm, or the like).

In this regard, the sensor device 106 and the external user device 108 can include suitable communication hardware and/or software that provides for wireless (and/or wired) communication the respective devices. For example, the sensor device 106 and the external user device 108 can be communicatively coupled via one or more networks (e.g., a personal area network (PAN), a local area network (LAN), a wide area network (WAN) such as the Internet, and the like). The sensor device 106 and the external user device 108 can employ various suitable wired and/or wireless communication technologies to communicate information therebetween. For example, some suitable communication technologies/protocols can include but are not limited to: Bluetooth®, Bluetooth low energy BTLE®, Mesh (e.g., IEEE 802.15.4), WiFi (e.g., IEEE 802.15.10), communication incorporating all or any portion of IEEE 802 or similar communication standards, RFID technology, near field communication (NFC), 3G communication, 4G communication, 5G communication, Backscatter communication, light communication, audio/sound communication, harvesting protocol communication (e.g., a metadata harvesting protocol), and the like. Other communications protocols or combinations of communications protocols (e.g., a Bluetooth/Mesh combined protocol) can be employed. Additionally, or alternatively, an acoustic or optical broadcasting can be employed. Although system 100 depicts a single external user device 108, it should be appreciated that the sensor device 106 can be configured to communicate with a plurality of external devices of varying types (e.g., user devices, routers, monitors, other sensor devices, server devices, cloud-based systems etc.).

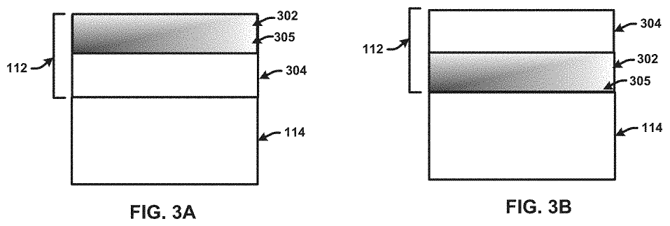

FIGS. 3A and 3B illustrate example indication units 112 (e.g., wet indication units 112) that provide a distinct optical signal 402 based on a combination of an optical property changing element 304 and a visual modifying element 302. In this regard, the indication unit 112 can include an optical property changing element 304 at least partially in overlapping relationship with a visual modifying element 302. In some nonlimiting examples as shown in FIG. 3A, the optical property changing element 304 can be positioned between the one or more internal layers 114 and the visual modifying element 302. As shown in FIG. 3B, the visual modifying element 302 can be positioned between the one or more internal layers 114 and the optical property changing element 304.

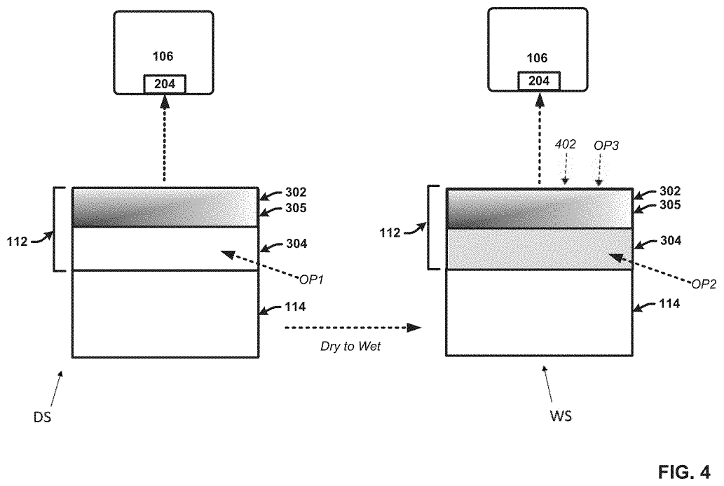

Turning to FIG. 4, the optical property changing element 304 can comprise an element that changes from having a first optical property OP1 to having a second optical property OP2 in response a chemical, biological and/or physical change to the article that indicates the presence of one or more bodily exudates. Optical properties include but are not limited to color, wavelength on the light spectrum, shape, size, saturation, transparency, hue, brightness and combinations thereof. The first and second optical property can vary with respect to any of the foregoing and/or other optical properties. It should be noted that changes in optical properties may not be detectable to the human eye but may be senses by the sensor device or other portions of the system.

The visual modifying element 302 can comprise an element proximate to the optical property changing element 304 that provides a visual modification to the second optical property, resulting in generation of a distinct optical signal 402 comprising a third optical property OP3. The distinct optical signal is based on the combination of the optical property changing element and the visual modifying element. The third optical property may be detected by the sensor device, and the sensor device itself and/or other portions of the system may determine the presence of bodily exudates or other characteristics related to usage activity based on the detection of the third optical property. Said determination may be made by processes in the sensor device, an external server device, external user device, cloud-based system, edge-based system or combinations thereof.

In the embodiment shown in FIG. 4, the backsheet 110 and the attachment zone 104 are removed for ease of illustration. As exemplified in FIG. 4, the sensor device 106 can monitor the visual state of the indication unit 112 over a course of wear of the absorbent article 102 using the detection unit 204. When the one or more internal layers 114 are dry (i.e., the dry state, DS), the optical property changing element 304 will have a first optical property. The optical property changing element 304 however will change to having a second optical property when the one or more internal layers 114 are wet (i.e., the wet state, WS), as illustrated by the change in coloration of the optical property changing element 304 when the internal layers 114 change from dry to wet.

For example, in one implementation, the optical property changing element 304 can include a conventional chemical based color-changing indicator that changes from a first color (e.g., yellow) to a second color (e.g., blue) based on the presence or a particular amount of bodily exudates. The visual modifying element 302 can further comprise an element or material layer 305 that changes the appearance of the second color, resulting in the generation of a distinct optical signal that comprises a third optical property. For example, in one implementation, the visual modifying element 302 can cause the second color to appear as a third color (e.g., purple) via a filtering effect. The sensor device 106 can further detect the presence of the bodily exudates in the absorbent article based on detection of the distinct optical signal comprising the third optical property (e.g., appearing purple) as opposed to the first or second optical property (e.g., appearing yellow or blue). In this regard, when bodily exudates are present within the one or more internal layers 114, the combination of the optical property changing element 304 and the visual modifying element 302 produces a unique optical signal that the sensor device 106 is specifically configured to detect.

The optical property changing element 304 can comprise an optical property changing composition or device. A color changing indicator can change its color, for example, based on the presence and/or absence of bodily exudates and/or in response to some other condition being monitored with respect to the absorbent article 102. In this regard, the optical property changing element 304 can include essentially any known color-changing indicator suitable for use in absorbent articles. For example, in some implementations, the optical property changing element 304 can include a color-changing indicator, such as a color strip, which comprises a chemical substance that can induce a color change in the color strip when bodily exudates are present within the one or more internal layers 114.

One useful form of a color-changing indicator comprises a pH-sensitive indicator. Bodily exudates, for example, may influence the pH-value in their environment. With these implementations, the color strip indicator can change color directly or indirectly in response to a change in pH within the absorbent article 102. For example, swelling of super absorbent polymers (SAP)/Absorbent Gelling Materials (AGM) within the absorbent article changes the pH of the environment within the absorbent article 102. In response to a change in pH, the color strip indicator can change in color. In one implementation, the color strip indicator can include a material (e.g., adhesive, SAP/AGM) that migrates within the article or fades as the more bodily exudates are introduced into the diaper.

Although a pH sensitive color strip indicator is discussed with respect to various example embodiments of the optical property changing element 304, the optical property changing element 304 is not limited to only color strip indicators. Rather, the optical property changing element 304 can include any indicator that changes color or other appearance directly or indirectly related to the presence or absence of bodily exudates within the one or more internal layers 114. For example, color change materials that change from no color to one or more colors, from one or more colors to no colors, change colors in other color ranges than the pH sensitive adhesive described herein, materials that change color or appearance based on factors other than pH changes, such as but not limited to, temperature, wetness, odor, enzymes, organic components, inorganic components (e.g., salt level), colored SAP/AGM, mechanical forces (e.g., strain, stretch) or the like.

The optical property changing element 304 can also comprise biological or physical sensor materials. For example, physical sensors can be provided by a material, which changes its color when the material is stretched. Stretching of a material can be induced by the swelling of the absorbent core, or other portions, of the absorbent article 102. Biological sensors can include a bioreceptor that interacts with an analyte of interest, such as trypsin or urease. A bioreceptor, for example, can use reagent/analyte interactions that provide a property change (e.g., a color or other optical change) in the optical property changing element 304 upon detection of a particular analyte of interest. Additionally, or alternatively, a bioreceptor can use an immobilized binding reagent also capable of binding to an analyte of interest. The immobilized reagent can be disposed within one or more material layers of the optical property changing element 304.

Additionally, or alternatively, the optical property changing element 304 can comprise a material selected from the group comprising, consisting essentially of or consisting of: thermochromic inks, thermochromic dyes, thermochromic liquid crystalline materials, and combinations thereof. These indicators can, for example, serve to monitor other conditions associated with the absorbent article and/or wearer, such as body temperature or fever indication.

As noted, the visual modifying element 302 comprises an element proximate to the optical property changing element that provides a visual modification to the second optical property generated by the optical property changing element 304, resulting in generation of a distinct optical signal comprising a third optical property. For example, in some implementations, the visual modifying element comprises a material 305 at least partially superposed the optical property changing element. The material 305 may be at least partially laid over (as shown in FIG. 3A) or under (e.g., as shown in FIG. 3B) the optical property changing element 304 that changes an appearance of the second optical property via a tinting or filtering effect. For example, in one or more implementations, the first optical property comprises a first color (e.g., yellow), the second optical property comprises a second color (e.g., blue), and the visual modification comprises a change in an appearance of the second color (e.g., a change in brightness, a change in coloration, a change in saturation, etc.).

In some implementations, the material 305 can comprise a thin layer of semitransparent material with a tinting property that causes the coloration of the optical property changing element 304 to appear different when the visual modifying element 302 overlaps at least a portion of the optical property changing element 304. Additionally, or alternatively, the visual modifying element 302 can comprise an ink or dye composition that is printed on the optical property changing element 304 that causes the coloration, saturation, brightness, etc., of the optical property changing element 304 to appear different when exposed to bodily exudates. In another implementation, the visual modifying element 302 can comprise a second optical property changing indicator (e.g., a second color changing indicator) that changes in a different manner than the optical property changing element 304 in response to presence of bodily exudates within the one or more internal layers 114. For example, assuming the optical property changing element 304 comprises a first color indicator that produces a first color change in response to exposure to bodily exudates (e.g., yellow to blue), the visual modifying element 302 can comprise a second color indicator that produces a different color change in response to the exposure to the bodily exudates (e.g., yellow to green, orange to red, etc.).

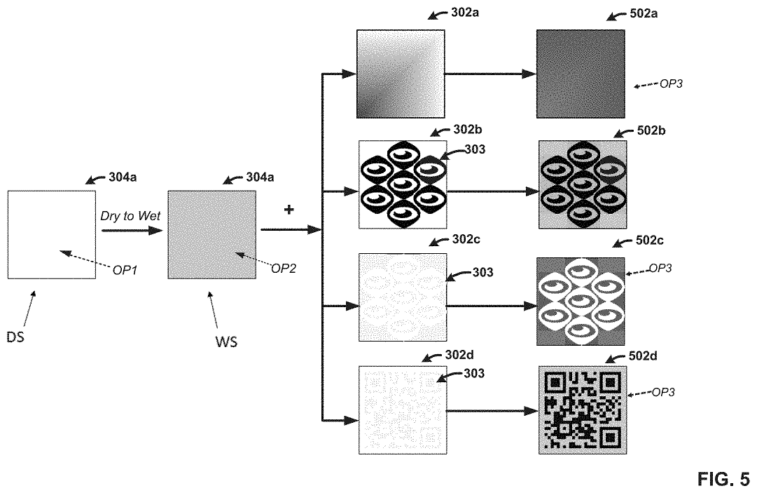

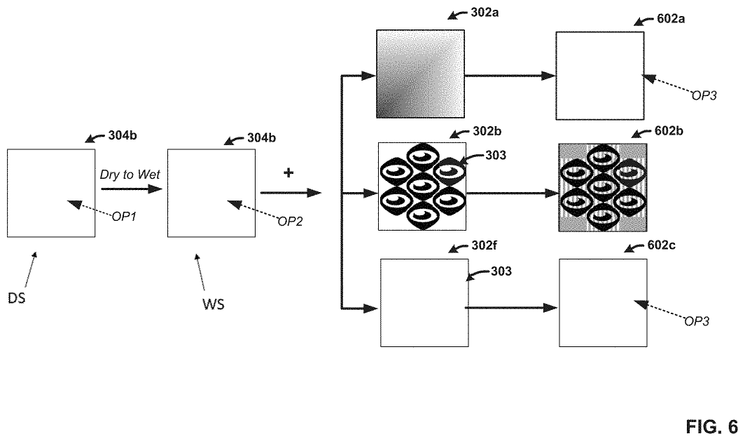

Turning to FIGS. 5-6, the visual modifying element 302 can comprise a defined visual mark 303. Nonlimiting examples of defined visual marks include a pattern, a symbol, an image, a code, a watermark and combinations thereof. The sensor device 106 can be configured to detect presence or absence of bodily exudates within the absorbent article based on detection of both the optical property change within the optical property changing element 304 and the visual mark 303 as well as any other visual modifications to the second optical property made by the visual modifying element (e.g., a third color change). For example, in one implementation, the visual modifying element 302 can include a unique visual mark printed on the optical property changing element 304. With this implementation, the sensor device 106 can be configured to detect presence or absence of bodily exudates based on detection of both the color change within the optical property changing element 304 and the printed visual mark. Still in other nonlimiting examples, the visual modifying element 302 and/or the optical property changing element 304 can be formed in the shape of a distinct pattern. With these embodiments, the distinct visual signal generated by the indication unit 112 can be based on a combination of the pattern of the visual modifying element 302 with the optical property changing element 304, an optical property change in the optical property changing element 304, and/or a visual modification to the optical property change as provided by the visual modifying element.

FIGS. 5 and 6 illustrate some example unique optical signals that can be generated by the indication unit 112 using different combinations of optical property changing elements and visual modifying elements.

In the embodiments shown in FIG. 5, the optical property changing element 304a can include a conventional chemical based indicator that changes from a first color (e.g., yellow) to a second color (e.g., blue or green) in response to exposure to bodily exudates (e.g., in the wet state). Various visual modifying elements respectively identified as visual modifying element 302a, visual modifying element 302b, visual modifying element 302c, and visual modifying element 302d are shown that can be combined with the optical property changing element 304a (e.g., overlapping and/or formed on at least a portion of the optical property changing element) to generate the corresponding distinct optical signals (e.g., respectively identified as distinct optical signal 502a, distinct optical signal 502b, distinct optical signal 502c and distinct optical signal 502d).

For example, in one implementation, the visual modifying element 302a can include tinted layer of semitransparent material or a printed layer of ink or dye formed on the optical property changing element 304a that causes the wet state optical property of the optical property changing element 304a to appear as a different coloration, as shown in the resulting distinct optical signal 502a. In another implementation, the visual modifying element 302b can include a defined mark 303 in the form of a unique pattern that is printed on or otherwise formed on the optical property changing element 304a. With this implementation, the resulting distinct optical signal 502b can comprise a combination of the wet coloration of the optical property changing element 304a and the unique pattern.

In another implementation, the visual modifying element 302c can include both a defined mark 303 in the form of a unique pattern that is printed on or otherwise formed on the optical property changing element 304a that also provides for changing an optical property of wet coloration (i.e., the second optical property) via a tinting or filtering effect, creating a third optical property. For example, the visual modifying element 302c consists of several circular eyelike symbols having a white coloration with a light shaded background color (e.g., light grey in this example). The combined effect of the wet coloration of the optical property changing element 304a and this visual modifying element 302c results in a distinct optical signal with a darkening of shade of the background color around the patterned eyelike symbols while the patterned eyelike symbols remain white. With this example, the white eyelike symbols can be formed with a solid non-transparent material that provides for blocking the underlying coloration of the optical property changing element 304a when wet, while the light-colored background of the visual modifying element 302c can comprise a material or dye that results in generation of a darker shade when combined with the wet coloration of the optical property changing element 304a, as shown in the resulting distinct optical signal 502c.

The combination of the wet coloration of the optical property changing element 304a and the visual modifying element 304d produces a similar type of effect to generate distinct optical signal 502d. With this example, the visual modifying element 302d can include a visual mark 303 in the form of a symbol or code, such as a QR code, that is faintly visible when formed on the optical property changing element 304a in the dry state, DS. The visual modifying element 302d however can include a reactive chemical composition and/or one or more tinted layers that when combined with the wet coloration of the optical property changing element 304a, causes the unique visual symbol to appear more distinct and detectable by the detection unit 204 of the sensor device 106.

FIG. 6 presents some additional example optical property changing element and visual modifying element combinations that can be used to generate distinct optical signals. In the embodiments shown in FIG. 6, the optical property changing element 304b can also include a conventional chemical based indicator that changes from a first color (e.g., yellow) to a second color (e.g., blue or green) in response to exposure to bodily exudates. However, different from the optical property changing element 304a, the second optical property OP2 of the optical property changing element 304b is formed in a pattern. It is also contemplated that the first optical property OP1 may be in the form of a pattern. With these embodiments, the wet state optical property OP2 can produce a first distinct visual pattern, which in this example consists of vertical stripes. When combined with various visual modifying elements, the resulting distinct optical signals are based on a combination of the first distinct visual pattern and the optical properties of the respective visual modifying elements. For example, in one implementation, when combined with visual modifying element 302a, the resulting distinct optical signal 602a includes a change (e.g., darkening) in coloration of the vertical stripes of the wet state of the optical property changing element 304b. In another example implementation, when combined with visual modifying element 302b, the resulting distinct optical signal 602b includes a defined visual mark 303 in the form of a pattern formed over the vertical stripes of the wet state of the optical property changing element 304b. In another example, when combined with visual modifying element 302f that consists of horizontal stripes, the resulting distinct optical signal 602c includes a checkered or grid pattern formed as a combination of vertical stripes and the horizontal stripes.

With reference now to FIG. 7, illustrated is a block diagram of an example sensor device (e.g., sensor device 106) configured to detect the presence of exudate in an absorbent article based on recognition of an optical signal generated by an indication unit of the absorbent article having a defined optical property.

In the embodiment shown, the sensor device 106 can include one or more sensors 702, a communication component 704, a detection unit 706, a notification component 708, at least one memory 712, at least one processor 714, a power unit 716 and a device bus 710. It should be appreciated that in some embodiments, one or more of these components/units can be removed from the sensor device 106. For example, in some embodiments, the detection unit 706 can include processing logic that provides for processing the measured sensor data (e.g., optical properties and/or optical signals of the indication unit) to determine a corresponding physical property of the absorbent article (e.g., a wetness level). In nonlimiting examples, some or all processing logic can be provided by and executed by an external device communicatively coupled to the sensor device (e.g., external user device 108). Repetitive description of like elements employed in respective embodiments is omitted for sake of brevity.

Various aspects of devices, systems, apparatuses or processes explained in this disclosure can constitute machine-executable component(s) embodied within machine(s), e.g., embodied in one or more computer readable mediums (or media) associated with one or more machines. Such components, when executed by the one or more machines (e.g., computers, computing devices, virtual machines, etc.), can cause the machines to perform the operations described. Accordingly, in one or more embodiments, the sensor device 106 can include at least one memory 712 configured to store computer executable components and instructions (e.g., the communication component 704, the detection unit 706, the notification component 708, etc.). The sensor device can also include at least one processor 714 (e.g., a microprocessor) to facilitate operation of the computer executable components and instructions by the sensor device 106. The sensor device 106 can further include a device bus 720 that couples the various components of the sensor device 106, including, but not limited to: one or more sensors 702, the communication component 704, the detection unit 706, the notification component 708, the at least one memory 712, the at least one processor 714, and the power unit 716.

With reference to FIG. 7 in view of FIGS. 1-6, the sensor device 106 can include one or more sensors 702 formed on or within a housing of the sensor device (e.g., housing 202) that are configured to sense, detect or otherwise capture sensor data regarding one or more physical properties or states of an absorbent article to which the sensor device is designed to be attached. the one or more sensors 702 can be or include any of the features and functionalities of any of the sensors 103, 203, 206 described above and vice versa. For example, in various embodiments, the one or more sensors 702 can include one or optical sensors 206 configured to detect information representative of presence or absence of bodily exudates, wetness levels, volume of bodily exudates, etc., based on optical responses/reactions reflected in the indication unit 112. In particular, in various embodiments described herein, the indication unit 112 can be particularly configured to generate a distinct optical signal indicative of the presence of exudates in the absorbent article (e.g., distinct optical signal 502a, distinct optical signal 502b, distinct optical signal 502c, distinct optical signal 502d, distinct optical signal 602a, distinct optical signal 602b, distinct optical signal 602c, and the like). The distinct optical signal is based on a combination of an optical property change in the optical property changing element 304 (e.g., a color change, a saturation level change, a brightness change, etc.) and the visual modifying element 302 as discussed above. For example, in some embodiments, the distinct optical signal can comprise a distinct color (e.g., purple) that is different from the color (e.g., blue or green) of the optical property changing element 304 alone when exposed to the bodily exudates. Additionally, or alternatively, the distinct optical signal can include a unique visual mark, such as a unique symbol, pattern, icon, code or the like.

In this regard, the one or more sensors 702 can include one or more optical sensors configured to detect and/or measure the distinct optical signal that indication unit 112 is configured to generate in response to exudates in the absorbent article 102. For example, in various embodiments, the one or more optical sensors can be configured to measure/detect optical property states/changes of the indication unit 112 including changes in coloration, brightness, saturation, and the like. For example, in implementations in which the indication unit 112 is configured to change color directly or indirectly in response to the presence of bodily exudates, the one or more optical sensors 206 can provide an output that varies depending on the presence or absence of bodily exudates which depends on an optical property (e.g., a color) measured by the one or more optical sensors.

In some embodiments, the one or more optical sensors can also be configured to measure variations in optical property states of the indication unit 112 that can be correlated to different physical properties/states (e.g., saturation levels, type of bodily exudates, volume of bodily exudates, biomarkers present in the bodily exudates, etc.). For example, while a human eye may only be able to distinguish general changes in color, such as a transition from yellow (dry) to blue (wet), an electronic color sensor such as a photodiode can detect intermediate colors as the indicator transitions from a first state to a second state. In some implementations, the sensor device 106 and/or the external user device 108 can further employ a detection algorithm to maintain a state of wet/soiled article once an initial detection has been made. For example, a ratchet or other feature in the detection algorithm can be used to maintain an indication that bodily exudates have been detected until the diaper has been changed or the sensor device 106 been removed from the diaper.

Some examples of optical sensors across a range of wavelengths are: electron tube detectors, photosensors, photomultiplier tubes, phototubes, photodetectors, opto-semiconductor detectors, photodiodes, photomultipliers, image sensors, infrared detectors, thermal sensors, illuminance sensors, visible light sensors and color sensors. In some implementations, the sensor device 106 can also include a light source, such as a light emitting diode (LED), organic light emitting diode (OLED), an incandescent light bulb, thermionic light emission, luminescence (e.g., among others, fluorescence, chemilluminescence, electroluminescence (e.g., LED), for emitting light onto an area, the wavelength or spectrum of which is to be assessed by the optical sensor. The optical sensor in some color detecting embodiments can be optimized for assessing a color of a color-changing indicator. The optical sensor can be sensitive to visible and non-visible light. In various embodiments, ultraviolet (UV), visible infrared and near infrared wavelengths may be used.

For example, with reference again to FIG. 2B, presented is example embodiment of a sensor device 106 including detection unit 204 that includes and/or is operatively coupled to an optical sensor 206 and a light source 208 (e.g., an LED) located on the backside of the sensor device 106. The detection unit 204 can include any of the features discussed with respect to the detection unit 706 in combination with one or more optical sensors of the one or more sensors 702, and vice versa. In one or more embodiments, the optical sensor 206 can be configured to measure one or more light levels of the indication unit 112 disposed within the absorbent article 102. In non-limiting examples, the optical sensor 206 can measure four light levels—clear, red, green and blue—with a sixteen (16) bit resolution. The clear level can correspond to a measure of an overall light intensity and the red, green and blue levels can correspond to intensity in the relevant parts of the spectrum from the indication unit 112. In this embodiment, the sensor device can take multiple measurements with the optical sensor 206. For example, in a first operation, the optical sensor 206 can be read without the light source 208 illuminated to determine a background light level. Another reading of the optical sensor 206 can also be taken in another operation with the light source 208 illuminating the indication unit 112 to measure the clear, red, green and blue (RGB) light levels. A difference between the two measurements is obtained in a third operation and represents a color of the indication unit 112. The clear color level can be used to normalize the RGB values. Saturation levels corresponding to one or more intermediate states of the indication unit 112 can also be determined, such as from the hue, saturation and brightness (HSB) values in combination with or instead of the RGB values.

The optical sensor 206 can be spaced from the light source 208 so that direct light from the light source 208 is reduced or eliminated at the optical sensor 206. Similarly, too large a spacing between the optical sensor 206 and the light source 208 can reduce the signal strength at the optical sensor 206. The optical sensor 206 can be spaced at least about 5.0 millimeters (mm), or at least about 8.0 mm, or at least about 10.0 mm, or from about 5.0 to about 20.0 mm, or from about 10.0 to about 15 mm from the light source 208, reciting for each range every 1 mm increment therein.

In addition to spacing between the optical sensor 206 and the light source 208, other factors may also affect light level measurements of the optical sensor 206. For example, temperature, location of the sensor device 106 on the article, the type, material and color of a connector (e.g., adhesive, tape, hook and loop, strap and other materials) disposed between the sensor device and the indicator, orientation of the sensor device 106 relative to the indicator, orientation of transmit and receive windows of the sensor device 106 and the article, force of application of the sensor device 106 against the article, ambient light, position of an attachment zone 104 on the article and position of the sensor device 106 relative to the indication unit 112 within the article (e.g., in a cross-direction) such that the optical sensor 206 detects other portions of the article disposed near the indication unit 112.

With reference again to FIG. 7 in view of FIGS. 1-6, one or more optical and/or image sensors configured to capture and/or detect visual characteristics of the indication unit 112 other than coloration properties (e.g., codes, patterns, marks). In various implementations, at least one sensor 702 can include a camera, an optical/image scanner (e.g., a barcode reader), an image sensor, and/or the like, that can capture and/or detect distinct visual patterns/identifiers in the indication unit 112. In this regard, the one or more sensors 702 can be configured to capture image data of the indication unit 112 at regular intervals over the course of wear and/or in response to a trigger event (e.g., a change in coloration of at least a part of the indication unit). The image data can then be processed by the sensor device 106 and/or the external user device 108 to determine whether the captured image data corresponds to the distinct optical signal indicative of wetness of the absorbent article (or another defined property/state of the absorbent article 102).

The communication component 704 can provide for communicatively coupling the sensor device 106 with one or more external devices, such as external user device 108, external sever device and/or various other devices remote (e.g., physically remote) from sensor device 106. In this regard, the communication component 704 can include software, hardware, or a combination of software and hardware that is configured to facilitate performance of wireless (and/or wired) communications between the sensor device and the one or more external devices. For example, the communication component 704 can include and/or be configured to control operation of one or more transmitters/receivers of the sensor device 106 to provide for transmitting information to the one or more external devices and/or receiving information from the one or more external devices.

The communication component 704 can be configured to facilitate wireless communication with the one or more external devices (e.g., the external user device 108 and/or other external devices) using a variety of wireless communication protocols. For example, in one or more embodiments, the communication component 704 can communicate with an external device using a Bluetooth® communication protocol, a near-field communication (NFC) protocol, or another type of communication protocol over a PAN or a LAN, (e.g., a Wi-Fi network) that can provide for communication over greater distances than NFC protocol or that can provide various advantages (such as increased security). Other communication protocols that can be employed by communication component 904 to communicate with an external device can include, but are not limited to: a Session Initiation Protocol (SIP) based protocol, a Zigbee® protocol, a RF4CE protocol, a WirelessHART protocol, a 6LoWPAN (IPv6 over Low power Wireless Personal Area Networks) protocol, a Z-Wave protocol, an ANT protocol, an ultra-wideband (UWB) standard protocol, a cellular communications protocol (e.g., second, third, fourth and fifth Generation Partnership Project (GGP) protocols, Long Term Evolution (LTE), protocols), machine type communication (MTC) protocols, Narrowband Internet-of-things (NB-IoT) protocols, other radio frequency (RF) communication protocols, non-RF communication protocols (e.g., induction based, optical based, audio based, etc.) and/or other proprietary and non-proprietary communication protocols.

In some embodiments, the communication component 704 can be configured to communicate captured/measured sensor data (e.g., captured via the one or more sensors 702) to an external device (e.g., external user device 108) for processing by the external device. For example, the communication component 704 can be configured to send some or all captured sensor data (e.g., sensor measurements, images, image signals, optical signals, etc.) to the external device, regardless as to whether the captured sensor data reflects a change in an optical state of the indication unit 112 and/or reflects occurrence of a distinct optical signal that corresponds to a monitored property of the absorbent article 102 (e.g., a wetness level). With these embodiments, the external device (e.g., the external user device 108) can include full onboard processing logic to analyze all or some of the captured sensor data for conversion to property/state conclusions about the absorbent article 102 or wearer of the absorbent article (e.g., the external device can process the received sensor data to determine whether the distinct optical signal has been provided by the indication unit 112).

The detection unit 706 can provide for full and/or partial processing of the sensor data captured via the one or more sensors 702 to generate sensory feedback data based on the captured sensor data regarding a corresponding state in the article (e.g., wetness level) or property associated with the absorbent article/wearer of the absorbent article. For example, in various embodiments, the detection unit 706 can be configured to process/analyze the sensor data captured via the one or more sensors using predefined processing logic (e.g., algorithms, heuristics, machine learning models, defined correlations, tracked data correlations, etc.) to determine and/or infer feedback information regarding urination and/or defecation events (e.g., including information regarding occurrence and timing of the events, volume of bodily exudates, saturation levels, loading status, type of the bodily exudates, biomarkers present in the bodily exudates, etc.). For example, in one or more implementations, an optical sensor of the sensor device 106 can detect and/or determine one or more RGB light levels or HSB levels of the indication unit 112. The detection unit 706 can further determine and/or infer sensory feedback data regarding presence, absence and/or amount of one or more bodily exudates, and/or a local saturation (e.g., exudate content quantity) level, based at least in part on the RGB light level or HSB level information as captured/generated by the one or more sensors 702. For example, the detection unit 706 can compare the RGB level or HSB level information to defined optical signal information stored in memory 712 of the sensor device 106 to determine whether the detected RBB level or HSB level information indicates the absorbent article has reached a defined state (e.g., wetness level).

In another example, the detection unit 706 can compare captured optical/image data of the indication unit 112 to defined information stored in memory 712 of the sensor device 106 that correlates one or more defined optical signals to a defined wetness level or another known property (e.g., exudate content quantity). For instance, the sensor device 106 can include information stored in memory 712 that identifies one or more distinct optical signals indicative of a defined wetness of the absorbent article (e.g., distinct optical signal 502a, distinct optical signal 502b, distinct optical signal 502c, distinct optical signal 502d, distinct optical signal 602a, distinct optical signal 602b, distinct optical signal 602c, and the like). With this implementation, the detection unit 706 can compare the captured sensor image/optical data with the defined optical signal data to determine a corresponding state of the absorbent article (e.g., to determine whether the absorbent article is wet or dry).

In some implementations of these embodiments, the communication component 704 can be configured to send the sensory feedback information to the external user device 108 for rendering and/or further processing by the external user device 108 and/or forwarding to an external server device (or another device). The external user device 108 can further be configured to present or otherwise provide the determined information to the user as a real-time notification (e.g., notification when diaper is wet), as an assessment report, or the like.

Additionally, or alternatively, the sensor device 106 can include a notification component that can be configured to generate and send notifications to the external device based on detection of defined sensor measurement values and/or based on a determination, by the detection unit 706, that a defined event or condition has occurred as determined based on the sensor data. For example, in some implementations, the notification component 708 can be configured to generate and send the external user device 108 a notification when the distinct optical signal provided by the indication unit 112 that has been detected by the detection unit 706. In another example, based on a determination that the optical state/signal provided by the indication unit 112 indicates the absorbent article has reached a defined level of saturation (e.g., as determined by the detection unit 706), the notification component 708 can send a notification to the external device indicating that the absorbent article is wet, needs to be changed or the like.

The sensor device 106 can further include a suitable power unit 717 to drive the functionality of the sensor device 106 and to provide power to the various electrical components of the sensor device 106. In one or more embodiments, the power unit 716 can include but is not limited to: a rechargeable battery, a non-rechargeable battery, a capacitor, a charge pump, a mechanically derived power source (e.g., microelectromechanical systems (MEMs) device), or an induction component. The sensor device 106 can also include various other device circuitry/hardware to facilitate operation of the various components of the sensor device 106. For example, the sensor device 106 can further include suitable electronic circuitry (e.g., hardware), software, or a combination thereof, that provides for processing of raw sensor measurements representative of a measured property (e.g., a wetness level, an activity level, etc.) as captured via the one or more sensors 702 of the sensor device 106 into a digital signal corresponding to the measured property. For example, such electronic circuitry can include but is not limited to, excitation control elements, amplification elements, analogue filtering elements, data conversion elements, compensation elements, and the like.

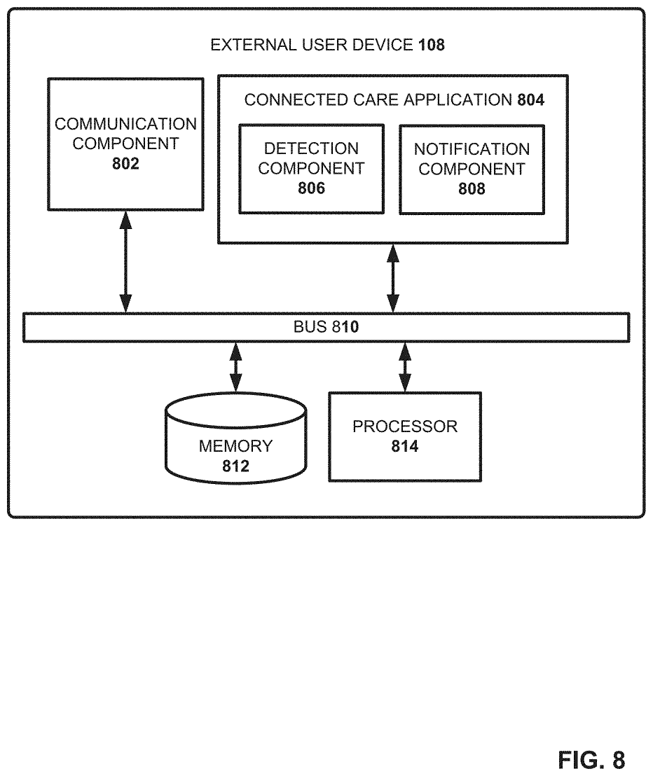

FIG. 8 illustrates a block diagram of an example external user device (e.g., external user device 108) facilitates determining and reporting a condition of an absorbent article based on recognition of an optical signal generated by an indication unit of the absorbent article having a defined optical property. In the embodiment shown, the external user device 108 can include various computer executable components associated with a connected care application 804 executed by the external user device 108. For example, the connected care application 804 can include a dedicated client application, a web-application, a thin client application, a hybrid application, or the like. For example, in some embodiments, the connected care application 804 can be configured to provide various features and functionalities associated with use of the sensor device 106 in association with communication with at least one external server device (e.g., a cloud-based server device, edge-based system, an application server, and the like).

In addition to the connected care application 804, the external user device 108 can include a communication component 802, at least one memory 812, at least one processor 814, and a device bus 810. In various embodiments, the at least one memory 812 can be configured to store computer executable components and instructions (e.g., the connected care application 804). The external user device 108 can also include at least one processor 814 to facilitate operation of the computer executable components and instructions by external user device 108. The external user device 108 can further include a device bus 810 that couples the various components of the external user device 108, including, but not limited to, the connected care application 804, the communication component 802, the memory 812, and the processor 814.

In the embodiment shown, the connected care application 804 can include detection component 806 and notification component 808. In some implementations, the detection component 806 can provide same or similar features and functionalities as the detection unit 706 of the sensor device. For example, in some implementations, the communication component 802 can received captured sensor data (e.g., captured via the one or more sensors 702) representative of an optical state/signal of the indication unit. With these implementations, the detection component 806 can process the sensor data using one or more algorithms, heuristics, and the like, to determine a corresponding property of the absorbent article 102 and/or wearer of the absorbent article. For example, the detection component 806 can compare the sensor data to defined optical signal information stored in memory 812 to determine whether the sensor data reflects occurrence of a distinct optical signal indicative of a particular condition of the absorbent article. The notification component 808 can also provide same or similar features and functionalities of notification component 708. For example, in some implementations, the notification component 808 can be configured to generate and/or render a notification at the external user device 108 (e.g., a visual notification, an audible notification, etc.) that informs the user regarding the usage of the absorbent article (e.g., presence of exudates) based on a determination that the sensor data indicates the distinct optical signal indicative has been emitted by the indication unit 112. The communication component 802 any of the features as communication component 704. Repetitive description of like elements employed in respective embodiments is omitted for sake of brevity.

The detection component 806 may determine sensory feedback information based on received sensor data provided by the sensor device 106. Additionally, or alternatively, the detection component 806 of the external user device 108 can further receive additional inputs, such as but not limited to: information regarding sensor device, the time the absorbent article was attached to a wearer, time the sensor device was attached to the absorbent article, the current time, wearer information (e.g., demographic information such as sex, age, weight of wearer, biometric information of the wearer, whether the wearer is toilet training, degree of wearer incontinence), temperature, humidity, caregiver preference information and/or ambient sensor information. The detection component may use these additional inputs separately and/or in combination with the information received from the sensor device.

With these embodiments, the detection component 806 can determine a predicted diaper fullness level (e.g., an exudate content quantity of the absorbent article such as a liquid and/or solid exudate content quantity) or state based at least in part upon the sensor data received from the sensor device 106 and contextual information (e.g., wear time of the diaper and baby demographic information). Contextual information, for example, can be input by consumers, retrieved via other sensors or information sources (e.g., thermostats). The data and inputs, for example, can include property change indications (e.g., digital or analog such as an intensity of color change in a color changing indicator) and wear time. In one implementation, an analog detection may be based upon a calibration of an indicator (e.g., color) for different exudate loads. Wear time, for example, may be described as the time determined between attachments of two fresh diapers. For example, in various implementations, the detection component 806 can determine and/or infer exudate fullness using one or more of the following functions: (Urine Fullness=f(Property Change Detection, wear time, wearer data and other data)); and Property Change Detection=f(color sensor data).

The notification component 808 can further report an exudate content quantity, for example, to a user to indicate a percent or other indication of diaper fullness or remaining capacity for displaying at the external user device 108. In one particular implementation, for example, the external user device 108 can display a graphical or numerical representation of exudate content quantity or remaining capacity of the absorbent article.

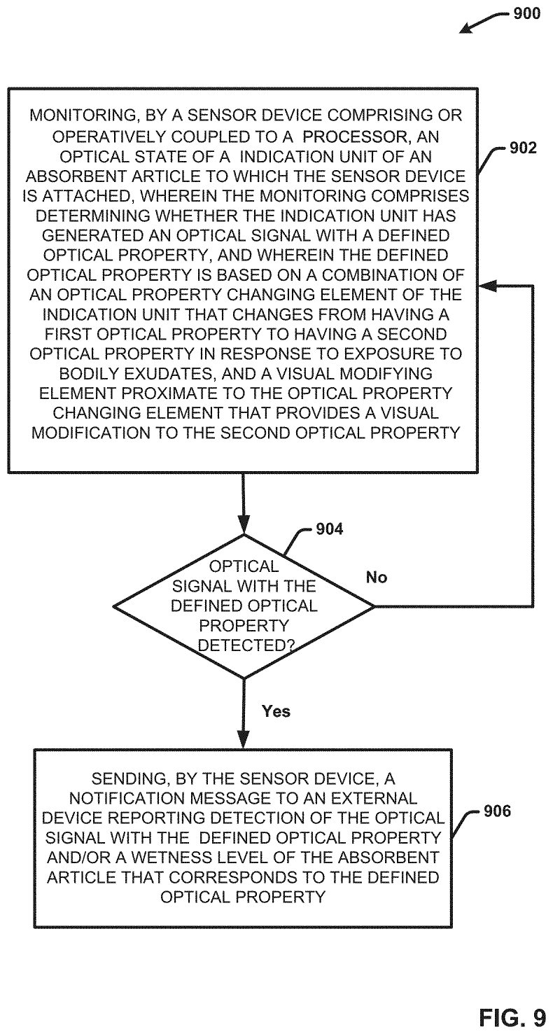

FIG. 9 presents a high-level flow diagram of another example process 900 for detecting a condition of an absorbent article based on recognition of an optical signal generated by an indication unit of the absorbent article having a defined optical property. Repetitive description of like elements employed in respective embodiments is omitted for sake of brevity.

At 902, a sensor device 106 comprising or operatively coupled to a processor can monitor (e.g., using one or more sensors 702 and/or the detection unit 706) an optical state of an indication unit 112 of an absorbent article to which the sensor device is attached. The monitoring comprises determining whether the indication unit has generated an optical signal with a defined optical property, and wherein the defined optical property is based on a combination of an optical property changing element 304 that changes from having a first optical property to having a second optical property in response to a change in the condition of the article (e.g., the presence of exudates), and a visual modifying element 302 proximate to the optical property changing element that provides a visual modification to the second optical property. At 904, the sensor device can determine whether the defined optical property has been detected. For example, in one implementation, using the detection unit 706, the sensor device can determine whether a distinct optical signal representative of a wet state of the absorbent article as defined in memory of the sensor device has been generated by the indication unit 112. If not, the sensor device can continue to monitor the indication unit for the distinct optical signal. However, if at 904, the sensor device determines that the optical signal with the distinct optical property has been detected, then at 906, the sensor device can send (e.g., using notification component 708 and/or communication component 704) a notification to an external device (e.g., external user device 108) reporting detection of the optical signal with the defined optical property and/or the condition of the absorbent article that corresponds to the defined optical property.

The present invention may be a system, a method, an apparatus and/or a computer program product at any possible technical detail level of integration. The computer program product can include a computer readable storage medium having computer readable program instructions thereon for causing a processor to carry out aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

The flowcharts and block diagrams in the Figures illustrate the architecture, functionality, and operation of possible implementations of systems, methods, and computer program products according to various embodiments of the present invention. The functions noted in the blocks can occur out of the order noted in the Figures. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the subject matter has been described above in the general context of computers, those skilled in the art will recognize that this disclosure also can or can be implemented in combination with other program modules. Generally, program modules include routines, programs, components, data structures, etc. that perform particular tasks and/or implement particular abstract data types. Moreover, those skilled in the art will appreciate that the inventive computer-implemented methods can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments in which tasks are performed by remote processing devices that are linked through a communications network. However, some, if not all aspects of this disclosure can be practiced on stand-alone computers. In a distributed computing environment, program modules can be located in both local and remote memory storage devices.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A system comprising:
an absorbent article that absorbs and contains bodily exudates; and
an indication unit formed on or within the absorbent article that generates an optical signal indicative of presence of the bodily exudates within the absorbent article, the indication unit comprising:
an optical property changing element that changes from having a first optical property to having a second optical property in response to exposure to the bodily exudates; and
a visual modifying element proximate to the optical property changing element that provides a visual modification to the second optical property, resulting in the optical signal comprising a third optical property.

2. The system of claim 1, wherein the visual modifying element comprises a material overlapping the optical property changing element that changes an appearance of the second optical property via a tinting and/or filtering effect.

3. The system of claim 1, wherein the first optical property comprises a first color and the second optical property comprises a second color, and wherein the visual modification comprises a change in an appearance of the second color.

4. The system of claim 3, wherein the visual modification causes the second color to appear as a third color.

5. The system of claim 1, further comprising a sensor device that determines the presence of the bodily exudates based on detection of the optical signal comprising the third optical property.

6. The system of claim 5, wherein the sensor device removably attaches to the absorbent article.

7. The system of claim 5, wherein the sensor device further sends a notification to an external device only in response to the detection of the optical signal comprising the third optical property.

8. The system of claim 1, wherein the visual modifying element comprises a defined visual mark, and wherein the system determines the presence of the bodily exudates based on a combination of the optical signal comprising the third optical property and the defined visual mark.

9. The system of claim 8, wherein the defined visual mark is selected from a group consisting of: a pattern, a symbol, an image, a code, a watermark and combinations thereof.

10. The system of claim 1, wherein an external server device, a cloud-based system, an edge-based system and/or an external user device determines the presence of the bodily exudates based on detection of the optical signal comprising the third optical property.

11. A sensor device comprising:
a detection unit that detects presence of bodily exudates in an absorbent article based on detection of an optical signal with a defined optical property generated by an indication unit of the absorbent article,
wherein the defined optical property is based on a combination of an optical property changing element of the indication unit that changes from having a first optical property to having a second optical property in response to exposure to the bodily exudates, and a visual modifying element proximate to the optical property changing element that provides a visual modification to the second optical property.

12. The sensor device of claim 11, wherein the first optical property comprises a first color and the second optical property comprises a second color, and wherein the visual modification comprises a change in an appearance of the second color.

13. The sensor device of claim 12, wherein the visual modification causes the second color to appear as a third color.

14. The sensor device of claim 11, wherein the visual modifying element comprises a defined visual mark, and wherein the detection unit further detects the presence of the bodily exudates based on the optical signal comprising the defined optical property and the defined visual mark.

15. The sensor device of claim 14, wherein the defined visual mark is selected from a group consisting of: a pattern, a symbol, an image, a code, a watermark and combinations thereof.

16. The sensor device of claim 11, further comprising a notification component that sends a notification to an external device only in response to the detection of the optical signal comprising the defined optical property.

17. A system comprising:
an absorbent article configured to absorb and contain bodily exudates;
an indication unit formed on or within the absorbent article, the indication unit comprising:
an optical property changing element that changes from having a first optical property to having a second optical property in response to exposure to the bodily exudates; and
a visual modifying element proximate to the optical property changing element that provides a defined visual mark; and
a sensor device that removably attaches to the absorbent article and detects presence of the bodily exudates in the absorbent article based on detection of a defined optical property that results from a combination of the second optical property and the defined visual mark.

18. The system of claim 17, wherein the sensor device further sends a notification to an external device only in response to the detection of the defined optical property.

19. The system of claim 17, wherein the first optical property comprises a first color and the second optical property comprises a second color, wherein the visual modifying element comprises a material that causes the second color to appear as a third color, and wherein the defined optical property comprises the third color and the defined visual mark.

20. The system of claim 17, wherein the defined visual mark selected from a group consisting of: a pattern, a symbol, an image, a code, a watermark and combinations thereof.
